(19) United States
(12) Patent Application Publication
(10) Pub. No.: US 2020/0179264 A1
(43) Pub. Date: Jun. 11, 2020

(54) AGENT AND METHOD FOR TEMPORARY DEFORMATION OF KERATIN-CONTAINING FIBERS

(71) Applicant: Henkel AG & Co. KGaA, Duesseldorf (DE)

(72) Inventors: ANNA PULS, Winsen (Luhe) (DE); SANDRA BRANDT, Pinneberg (DE); ARNE JUNGE, Hamburg (DE)

(73) Assignee: Henkel AG & Co. KGaA, Duesseldorf (DE)

(21) Appl. No.: 16/711,270

(22) Filed: Dec. 11, 2019

(30) Foreign Application Priority Data
Dec. 11, 2018 (DE) 10 2018 221 409.3

(51) Int. Cl. A61K 8/891; A61K 8/19; A61K 8/34; A61K 8/25; A61Q 5/06

(52) U.S. Cl. CPC A61K 8/891; A61K 8/19; A61Q 5/06; A61K 8/25; A61K 8/345

(57) ABSTRACT

Powdery cosmetic compositions containing, based on their total weight

a) from about 5 to about 20% by weight of hydrophobized metal oxide powder

b) from about 40 to about 94% by weight of organic polyol

c) from about 0.1 to about 15% by weight of emulsifier

d) from about 0.1 to about 10% by weight of composite powder, including

(A) powder particles of a silicone elastomer and

(B) particles of silicon dioxide,

wherein the particles of silicon dioxide (B) are immobilized on the surface of the powder particles of the silicone elastomer (A), and

e) from 0 to about 40% by weight of water,

are particularly suitable for the deformation of keratinic fibers, imparting shine, texture and suppleness, without creating a sticky feeling on the skin and/or the keratinic fibers.

### CROSS-REFERENCE TO RELATED APPLICATION

This application claims priority to German Patent Application No. 10 2018 221 409.3, filed Dec. 11, 2018, which is incorporated herein by reference in its entirety.

### TECHNICAL FIELD

The application relates to the technical field of temporary deformation keratin-containing fibers, in particular human hair.

### BACKGROUND

Styling agents for the deformation of keratin-containing fibers have been known a long time and are used in various embodiments to build, refresh and fix hairstyles with many hair types that can only be obtained using firming active substances. In this case, both hair treatment agents, which are used for a permanent and a temporary shaping of the hair, play an important role. The temporary shaping, which are intended give a good hold without impairing the healthy appearance of the hair, such as its shine, can be achieved, for example, by hair sprays, hair waxes, hair gels, hair waves, etc.

Corresponding agents for temporary shaping usually contain synthetic polymers as the shaping component. Preparations containing a polymer can be applied to the hair by employing propellant gases or by a pumping mechanism. Hair gels and hair waxes, however, are usually not applied directly to the hair, but distributed by employing a comb or the hands into the hair.

Known forms of temporary styling agents often cannot be dosed with satisfactory accuracy. Thus, hair gels, hair creams and hair waxes are difficult to distribute once they are applied to the hair.

Hair sprays can be distributed more evenly on the hair. However, since the user has no ability to visually detect the total amount of styling agent applied, there is a risk that more styling agent will be applied to the hair than would actually be required.

Powdered cosmetics are known and long been used, for example, in the field of skin treatment. Typical examples are make-up powder or eye shadow. The use of a powdered carrier material is required in order to achieve the powdered consistency. A metal oxide such as silicon dioxide can be used as a suitable carrier material. Particularly interesting is hydrophobized metal oxide or silicon dioxide. This can be obtained, for example, starting from fumed silicon dioxide, which is commercially available in various specifications. Untreated fumed silicon dioxide carries silanol and siloxane groups on the surface. As a result, it has a high affinity to water, that is, it is hydrophilic. Alkylsilyl groups can be chemically bonded to the surface of the fumed silicon dioxide by reaction with suitable organic silicon compounds. This results in modified silicon dioxide powder, which is no longer wetted by water, that is, having hydrophobic properties. These hydrophobized silicon dioxides are suitable for the production of so-called dry water, in which the water droplets are prevented from flowing together. The resulting powdered solids can have water content of up to about 95%. The enclosed water is released again under mechanical stress, for example, rubbing on the skin.

Cosmetic or pharmaceutical liquefiable powder compositions are described, for example, in European Patent EP 1 235 554 B1.

International application WO 03/037287 A1 discloses the use of granules based on fumed silicon dioxide in cosmetic compositions. The specific granules can be silanized, that is, hydrophobized and are suitable for the production of cosmetic compositions of any consistency, for example, liquids, foams, sprays or powders.

The international application WO 2007/051511 A1 describes the use of a powdered composition containing 50 to 95% by weight of an aqueous solvent, hydrophobized silicon dioxide powder and at least one film-forming and/or firming polymer in the aqueous solvent for the temporary deformation of keratinic fibers.

The German patent application DE102008057261 A1 has as the subject powdered compositions which are used for the temporary forming of hair for very strong hold of the fixed hairstyle.

DE 102012211912 A1 discloses powdered cosmetic compositions having hydrophobized metal oxide powder, organic polyol and an emulsifier.

A sticky feeling often sets in when rubbing such powdered compositions on the hand. Also, powdered compositions often impart a rough and thereby unnatural feel to the hair.

### BRIEF SUMMARY

Powdered cosmetic compositions and methods for the temporary deformation of keratin-containing fibers are provided herein. In an embodiment, a powdered cosmetic composition includes, based on its total weight

a) from about 5 to about 20% by weight of hydrophobized metal oxide powder,

b) from about 40 to about 94% by weight of organic polyol,

c) from about 0.1 to about 15% by weight of emulsifier,

d) from about 0.1 to about 10% by weight of composite powder, comprising
(A) powder particles of a silicone elastomer and
(B) particles of silicon dioxide,
wherein the particles of silicon dioxide (B) are immobilized on the surface of the powder particles of the silicone elastomer (A), and

e) from 0 to about 40% by weight of water.

In another embodiment, a method is provided for the temporary deformation of keratin-containing fibers, in which the keratinic fibers are acted upon by a cosmetic composition and temporarily fixed in their form. The cosmetic composition includes, based on its total weight

a) from about 5 to about 20% by weight of hydrophobized metal oxide powder,

b) from about 40 to about 94% by weight of organic polyol,

c) from about 0.1 to about 15% by weight of emulsifier,

d) from about 0.1 to about 10% by weight of composite powder, comprising
(A) powder particles of a silicone elastomer and
(B) particles of silicon dioxide,
wherein the particles of silicon dioxide (B) are immobilized on the surface of the powder particles of the silicone elastomer (A), and

e) from 0 to about 40% by weight of water.
A plastically deformable mass is formed from the cosmetic composition by action of a force before, during or after application to the keratinic fibers.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure was therefore to provide a storage-stable powdered composition for the temporary shaping of hair that can be accurately and easily dosed, the hair not sticking together, imparting a fuller and natural and pleasant feel and natural shine to the hair. The durability of the styling result should not be impaired. In addition, the powdered cosmetic compositions should have improved feel, in particular reduced tack, when applied by hand.

It has been found that the addition of a selected composite powder to powdered cosmetic compositions based on a polyol-emulsifier mixture solves the aforementioned object. A first subject of the present application is powdered cosmetic compositions containing, based on their total weight,
a) from about 5 to about 20% by weight of hydrophobized metal oxide powder,
b) from about 40 to about 94% by weight of organic polyol,
c) from about 0.1 to about 15% by weight of emulsifier,
d) from about 0.1 to about 10% by weight of composite powder, including
(A) powder particles of a silicone elastomer and
(B) particles of silicon dioxide,
wherein the particles of silicon dioxide (B) are immobilized on the surface of the powder particles of the silicone elastomer (A), and
e) from 0 to about 40% by weight of water.

The powdered compositions are preferably in the form of core-shell particles, the shell of which contains particles of at least one hydrophobized metal oxide powder and the emulsifier and the liquid core of which contains organic polyol and optionally water.

In the context of the present disclosure, particles are particles which are present as grains (see DIN 66160: 1992-09) of solids.

In the context of the present disclosure, powdered are compositions whose particles are freely flowable under their own weight (see DIN EN ISO 6186: 1998-08).

The powdered compositions are distinguished by the fact that the liquid core is released from the core-shell particles by mechanical loading of the core-shell particles, in particular by friction and/or pressure, thereby forming a liquid from the powdered composition. It is thus a powdered powder-to-liquid composition. The powdered compositions can be dosed very easily. They can also be very evenly distributed in the hair, since the liquid core is released only under mechanical stress at the site of action and a targeted wetting of the hair fibers is possible. The powder can thus be first carefully distributed in the hair and only then subjected to mechanical stress, for example, by targeted massaging of the powder into the hair. As a result, the styling effect only unfolds directly on the desired hair part. Alternatively, the powdered composition can first be dosed into the hand, liquefied by mechanical stress (for example, by rubbing) and then applied to the hair.

Surprisingly, it has been shown that the addition of a composite powder including powder particles of a silicone elastomer and SiO₂ particles improves the product feel on the hand and on/in the hair without adversely affecting the stability or the styling performance of the powdered composition.

The powdered compositions used contain hydrophobized metal oxide. Preferred compositions contain the hydrophobized metal oxide powder in amounts of from about 5.5 to about 18, preferably from about 6 to about 15% by weight, based on its total weight. The optimum amount in this case depends primarily on the hydrophobicity of the silicon dioxide powder used. The more hydrophobic the silicon dioxide powder is, the less of it is needed to obtain a stable, powdered product.

The nature of the hydrophobized metal oxide is not limited in principle, as long as it is ensured that a powdered product is created when there is intensive mixing with the liquid, aqueous phase. In the context of the present disclosure, hydrophobized are to be understood to mean those metal oxides which have been modified at least on the surface of the particles in such a way that the modified particle is wetted less by water than the unmodified particle. In particular, silanized, hydrophobized metal oxides are preferred. At least one member of the group which is formed from silanes, halosilanes, alkoxysilanes and silazanes is preferably suitable as the reagent for silanizing the metal oxide. Preferably suitable hydrophobized metal oxides of the hydrophobized metal oxide powder are selected from at least one member of the group formed from hydrophobized silicates, hydrophobized aluminum silicates, hydrophobized titanium dioxide and hydrophobized silicon dioxide. Hydrophobized silicates have proven particularly suitable for the preparation of the powdered cosmetic compositions, wherein fumed silica aftertreated with polydimethylsiloxane has particular advantages. Preferred powdered compositions contain a hydrophobized silicate as hydrophobized metal oxide powder, preferably a fumed silica aftertreated with polydimethylsiloxane. Corresponding metal oxides with the INCI designation “Silica Dimethicone Silylate” are sold, for example, by Evonik under the trade name Aerosil® R202.

The particle diameter of the primary particles of preferred hydrophobized metal oxides is preferably less than about 5 μm, particularly preferably less than about 1 μm, and in particular between about 1 and about 50 nm.

Preference is furthermore given to those hydrophobized silicon dioxides which have a BET specific surface area of between about 10 and about 400 m²/g, preferably between about 40 and about 300 m²/g and in particular between about 80 and about 150 m²/g.

The powdered compositions contain an organic polyol as a second essential constituent. Preferred compositions contain the organic polyol based on their total weight in amounts of from about 45 to about 92% by weight, preferably from about 60 to about 90% by weight and in particular from about 65 to about 88% by weight. The polyol can be used as a single substance or in the form of polyol mixtures. Preferred compositions contain less than four, preferably one to three, polyol(s). Polyols from the group of glycerol, 1,2-ethanediol, polyethylene glycols having a molecular weight >400, propanediol, butanediol, in particular 1,3-butanediol, hexanediol, in particular 1,6-hexanediol, sorbitol, threitol, erythritol, arabitol, altritol, ribitol, xylitol, galactitol, mannitol, iditol and panthenol are in particular suitable for the preparation of cosmetic compositions. Particularly suitable organic polyols are glycerol, sorbitol and panthenol. These polyols can be converted into a powder form by comparatively small amounts of hydrophobized metal oxide powder, generally containing less than about 10% by weight of metal oxide powder (based on the total weight of the powdered cosmetic composition). Preferred compositions contain at least one compound from the group of glycerol, sorbitol and panthenol as an organic polyol.

A further essential constituent of the compositions is an emulsifier. Preferred compositions contain the emulsifier based on their total weight in amounts of from about 1 to about 12% by weight, preferably from about 2 to about 11% by weight and in particular from about 5 to about 10% by weight.

The cosmetic agents preferably further comprise at least one member selected from the group of anionic, amphoteric, zwitterionic, nonionic, cationic emulsifiers or mixtures thereof. Preference is given to using emulsifiers solid at room temperature (20° C.), in particular solid anionic emulsifiers at room temperature (20° C.).

Anionic emulsifiers are exemplified by a hydrophilic anionic group, for example, a carboxylate, sulfate, sulfonate or phosphate group and a lipophilic alkyl group. In addition, glycol or polyglycol ether groups, ester, ether and amide groups and hydroxyl groups can be present in the molecule. Examples of suitable anionic surfactants and emulsifiers are (in each case in the form of the sodium, potassium and ammonium as well as the mono-, di- and trialkanolammonium salts having from about 2 to about 4 C atoms in the alkanol group):
linear and branched fatty acids having from about 8 to about 30 carbon atoms (soaps),
ether carboxylic acids of the formula R—O—(CH₂—CH₂O)ₓ—CH₂—COOH, in which R is a linear alkyl group having from about 8 to about 30 C atoms and x=0 or 1 to 16,
acylsarcosides having from about 8 to about 24 carbon atoms in the acyl group,
acyltaurides having from about 8 to about 24 carbon atoms in the acyl group,
acyl isethionates having from about 8 to about 24 carbon atoms in the acyl group,
linear alkanesulfonates having from about 8 to about 24 C atoms,
linear alpha-olefin sulfonates having from about 8 to about 24 C atoms,
Alpha-sulfofatty acid methyl esters of fatty acids having from about 8 to about 30 carbon atoms,
acylglutamates, in particular sodium N-cocoyl and sodium N-stearoyl-L-glutamate,
   esters of sulfosuccinic acid or sulfosuccinates of the general formula (T3),

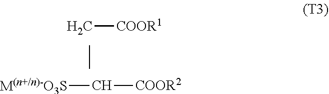

in which M^{(n+/n)} for n=1, represents a hydrogen atom, an alkali metal cation, an ammonium group or the cation of an ammonium organic base and for n=2, represents an alkaline earth metal cation and R¹ and R² independently of one another mean a hydrogen atom, an alkali metal cation or alkaline earth metal cation, an ammonium group, the cation of an ammonium organic base or a radical Z derived from a polyhydroxylated organic compound selected from the group of etherified (C₆-C₁₈) alkyl polysaccharides having from about 1 to about 6 monomeric saccharide units and/or the etherified aliphatic (C₆-C₁₆) hydroxyalkyl polyols having from about 2 to about 16 hydroxyl radicals which are etherified, with the proviso that at least one of the groups R¹ or R² is a radical Z,
sulfosuccinic acid mono- and dialkyl esters having from about 8 to about 24 C atoms in the alkyl group and sulfosuccinic monoalkylpolyoxyethyl esters having from about 8 to about 24 C atoms in the alkyl group and 1 to 6 oxyethyl groups,
alkyl sulfates and alkyl polyglycol ether sulfates of the formula R—(O—CH₂—CH₂)ₓ—OSO₃H, in which R is a preferably linear alkyl group having from about 8 to about 30 C atoms and x=0 or 1-12,
mixed surface-active hydroxysulfonates according to DE-A-37 25 030,
esters of tartaric acid and citric acid with alcohols which represent addition products of about 2-15 molecules of ethylene oxide and/or propylene oxide onto C₈₋₂₂ fatty alcohols,
alkyl and/or alkenyl ether phosphates,
sulfated fatty acid alkylene glycol esters,
monoglyceride sulfates and monoglyceride ether sulfates.

Particularly preferred anionic emulsifiers are Cetearyl Sulfosuccinate (INCI) and Stearoyl Glutamate (INCI).

In summary, preferred emulsifier emulsifiers contain an emulsifier solid at room temperature (20° C.) as an emulsifier, particularly preferably an anionic emulsifier solid at room temperature (20° C.), in particular at least one emulsifier from the group of Cetearyl Sulfosuccinate (INCI) and Stearoyl Glutamate (INCI). Particularly preferred anionic emulsifiers comprise Disodium Cetearyl Sulfosuccinate (INCI) and Sodium Stearoyl Glutamate (INCI).

The powdered compositions contain a selected composite powder as a fourth essential constituent. The composite powder includes, and may consist of, (A) powder particles of a silicone elastomer and (B) particles of silicon dioxide. The particles of silicon dioxide (B) are immobilized on the surface of the powder particles of the silicone elastomer (A).

The composite powders are extremely flowable, easily dispersible and have only a very low aggregation tendency. They can be incorporated into cosmetic compositions without special devices or special method steps. In general, it is sufficient to mix the composite powder with the other constituents.

Preference is given to composite powders, the powder particles of which have an average particle diameter of from about 0.5 to about 100 μm, more preferably from about 1 to about 10 μm.

The powder particles of the silicone elastomer (A) can have different shapes, wherein spherical powder particles are preferred.

The powdered silicone elastomer (A) can preferably be prepared by reacting an organopolysiloxane (I) having at least two alkenyl groups per molecule with an organopolysiloxane (II) having at least two hydrogen atoms bonded to a silicon atom per molecule.

In this case, a compound of the formula (I) is preferably used as an organopolysiloxane (I) which has at least two alkenyl groups per molecule

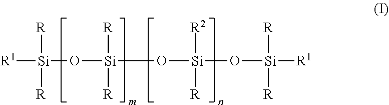

wherein

R stands for a C₁-C₆ alkyl group or a C₆-C₁₀ aryl group,

R¹ stands for R or an alkenyl group,

R² stands for R or an alkenyl group and

m and n are each independently of one another an integer from 0 to about 5,000, with the proviso that in the case where R¹ does not stand for an alkenyl group, R² means an alkenyl group and n is at least about 2.

a compound of the formula (II) is preferably used as an organopolysiloxane (II) having at least two hydrogen atoms bonded to a silicon atom per molecule

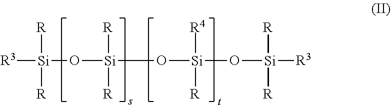

wherein

R has the above meaning,

R³ stands for R or hydrogen,

R⁴ stands for R or hydrogen and

s and t are each independently of one another an integer from 0 to about 5,000, with the proviso that in the case where R³ does not stand for hydrogen, R⁴ stands for hydrogen and t is at least about 2.

For example, methyl, ethyl, propyl and isopropyl can be mentioned as a C₁-C₆ alkyl group, wherein methyl is preferred. Suitable C₆-C₁₀ aryl groups are, for example, phenyl and naphthyl, wherein phenyl is preferred. More preferably, R stands for methyl in the above formulas,

C₂-C₁₀ alkenyl, such as vinyl, allyl, propenyl, butenyl, pentenyl, hexenyl and decenyl can be mentioned by way of example as an alkenyl group. The alkenyl group is preferably vinyl.

m, n, s and t each independently of one another preferably stand for an integer from 0 to about 1000, more preferably from 0 to about 200.

Preferably, the powdered silicone elastomer (A) is a dimethicone-vinyldimethicone copolymer, preferably a crosslinked dimethicone-vinyldimethicone copolymer. The latter are known by the INCI name Dimethicone/Vinyl Dimethicone Crosspolymer.

The particles of silicon dioxide (B) preferably have an average particle diameter of from about 0.001 to about 0.1 μm.

The silicon dioxide (B) is preferably amorphous and can be prepared by known methods, for example, in an electric arc, wet-chemical or by flame hydrolysis. Preferably, the silicon dioxide (B) is fumed silica.

For the immobilization of the silicon dioxide (B) on the powdered silicone elastomer (A), the latter can be converted, for example, into an aqueous dispersion to which the silicon dioxide (B) is added. The quantitative ratio can be chosen freely within wide limits, it being preferred to add from about 0.1 to about 30 parts by weight of silicon dioxide (B) to about 100 parts by weight of powdered silicone elastomer (A). The dispersion is heated, wherein the preferred temperature range is from about 40 to about 95° C. Finally, the water is removed and the composite powder thus obtained.

Particular preference is given to using the commercial product Dow Corning 9701 Cosmetic Powder having the INCI name Dimethicone/Vinyl Dimethicone Crosspolymer from Dow (and) Silica as the composite powder d).

The amount of composite powder d) in the powdered, cosmetic composition is preferably from about 0.5 to about 9% by weight, more preferably from about 1 to about 8% by weight and particularly preferably from about 2 to about 6% by weight, each based on the total weight of powdered, cosmetic composition.

Finally, the powdered cosmetic compositions can also contain water. However, the preparation of low-water compositions has proved to be advantageous for the cosmetic properties of the powdered composition.

However, the amount of water in the powdered compositions can also depend in particular on the type of polyol used. For example, powdered compositions having high levels of sorbitol and/or panthenol contain higher levels of water than, for example, powdered compositions having a high content of glycerol.

The composition of some preferred powdered, cosmetic compositions can be found in the following table (specifications as active substance content in % by weight relative to the total weight of the powdered composition, unless stated otherwise).

| | Formula | Formula | Formula | Formula | Formula |
|---|---|---|---|---|---|
| | 1 | 2 | 3 | 4 | 5 |
| Hydrophobized metal | 5 to 20 | 5.5 to 18 | 6 to 15 | 6 to 10 | 6 to 10 |
| oxide powder | | | | | |
| Polyol | 40 to 94 | 45 to 92 | 60 to 90 | 65 to 88 | 65 to 88 |
| Emulsifier | 0.1 to 15 | 1 to 12 | 2 to 11 | 5 to 10 | 5 to 10 |
| Composite powder* | 0.1 to 10 | 0.5 to 9 | 1 to 8 | 2 to 6 | 2 to 6 |
| Water | 0 to 40 | 0 to 25 | 0 to 10 | <1 | <0.5 |
| | Formula | Formula | Formula | Formula | Formula |
| | 6 | 7 | 8 | 9 | 10 |
| Silica Dimethicone | 5 to 20 | 5.5 to 18 | 6 to 15 | 6 to 10 | 6 to 10 |
| Silylate | | | | | |
| Polyol | 40 to 94 | 45 to 92 | 60 to 90 | 65 to 88 | 65 to 88 |
| Emulsifier | 0.1 to 15 | 1 to 12 | 2 to 11 | 5 to 10 | 5 to 10 |
| Composite powder* | 0.1 to 10 | 0.5 to 9 | 1 to 8 | 2 to 6 | 2 to 6 |
| Water | 0 to 40 | 0 to 25 | 0 to 10 | <1 | <0.5 |
| | Formula | Formula | Formula | Formula | Formula |
| | 11 | 12 | 13 | 14 | 15 |
| Hydrophobized metal | 5 to 20 | 5.5 to 18 | 6 to 15 | 6 to 10 | 6 to 10 |
| oxide powder | | | | | |
| Glycerol | 40 to 94 | 45 to 92 | 60 to 90 | 65 to 88 | 65 to 88 |
| Emulsifier | 0.1 to 15 | 1 to 12 | 2 to 11 | 5 to 10 | 5 to 10 |
| Composite powder* | 0.1 to 10 | 0.5 to 9 | 1 to 8 | 2 to 6 | 2 to 6 |
| Water | 0 to 40 | 0 to 25 | 0 to 10 | <1 | <0.5 |
| | Formula | Formula | Formula | Formula | Formula |
| | 16 | 17 | 18 | 19 | 20 |
| Silica Dimethicone | 5 to 20 | 5.5 to 18 | 6 to 15 | 6 to 10 | 6 to 10 |
| Silylate | | | | | |
| Glycerol | 40 to 94 | 45 to 92 | 60 to 90 | 65 to 88 | 65 to 88 |
| Composite powder* | 0.1 to 10 | 0.5 to 9 | 1 to 8 | 2 to 6 | 2 to 6 |
| Emulsifier | 0.1 to 15 | 1 to 12 | 2 to 11 | 5 to 10 | 5 to 10 |
| Water | 0 to 40 | 0 to 25 | 0 to 10 | <1 | <0.5 |
| | Formula | Formula | Formula | Formula | Formula |
| | 21 | 22 | 23 | 24 | 25 |
| Hydrophobized metal | 5 to 20 | 5.5 to 18 | 6 to 15 | 6 to 10 | 6 to 10 |
| oxide powder | | | | | |
| Sorbitol | 40 to 94 | 45 to 92 | 60 to 90 | 65 to 88 | 65 to 88 |
| Emulsifier | 0.1 to 15 | 1 to 12 | 2 to 11 | 5 to 10 | 5 to 10 |
| Composite powder* | 0.1 to 10 | 0.5 to 9 | 1 to 8 | 2 to 6 | 2 to 6 |
| Water | 10 to 40 | 10 to 30 | 10 to 30 | 15 to 30 | 15 to 30 |
| | Formula | Formula | Formula | Formula | Formula |
| | 26 | 27 | 28 | 29 | 30 |
| Silica Dimethicone | 5 to 20 | 5.5 to 18 | 6 to 15 | 6 to 10 | 6 to 10 |
| Silylate | | | | | |
| Sorbitol | 40 to 94 | 45 to 92 | 60 to 90 | 65 to 88 | 65 to 88 |
| Emulsifier | 0.1 to 15 | 1 to 12 | 2 to 11 | 5 to 10 | 5 to 10 |
| Composite powder* | 0.1 to 10 | 0.5 to 9 | 1 to 8 | 2 to 6 | 2 to 6 |
| Water | 10 to 40 | 10 to 30 | 10 to 30 | 15 to 30 | 15 to 30 |
| | Formula | Formula | Formula | Formula | Formula |
| | 31 | 32 | 33 | 34 | 35 |
| Hydrophobized metal | 5 to 20 | 5.5 to 18 | 6 to 15 | 6 to 10 | 6 to 10 |
| oxide powder | | | | | |
| Panthenol | 40 to 94 | 45 to 92 | 60 to 90 | 65 to 88 | 65 to 88 |
| Emulsifier | 0.1 to 15 | 1 to 12 | 2 to 11 | 5 to 10 | 5 to 10 |
| Composite powder* | 0.1 to 10 | 0.5 to 9 | 1 to 8 | 2 to 6 | 2 to 6 |
| Water | 10 to 40 | 10 to 25 | 12 to 25 | 15 to 25 | 15 to 25 |
| | Formula | Formula | Formula | Formula | Formula |
| | 36 | 37 | 38 | 39 | 40 |
| Silica Dimethicone | 5 to 20 | 5.5 to 18 | 6 to 15 | 6 to 10 | 6 to 10 |
| Silylate | | | | | |
| Panthenol | 40 to 94 | 45 to 92 | 60 to 90 | 65 to 88 | 65 to 88 |
| Emulsifier | 0.1 to 15 | 1 to 12 | 2 to 11 | 5 to 10 | 5 to 10 |
| Composite powder* | 0.1 to 10 | 0.5 to 9 | 1 to 8 | 2 to 6 | 2 to 6 |
| Water | 10 to 40 | 10 to 25 | 12 to 25 | 15 to 25 | 15 to 25 |
| | Formula | Formula | Formula | Formula | Formula |
| | 41 | 42 | 43 | 44 | 45 |
| Hydrophobized metal | 5 to 20 | 5.5 to 18 | 6 to 15 | 6 to 10 | 6 to 10 |
| oxide powder | | | | | |
| Polyol | 40 to 94 | 45 to 92 | 60 to 90 | 65 to 88 | 65 to 88 |
| Anionic emulsifier | 0.1 to 15 | 1 to 12 | 2 to 11 | 5 to 10 | 5 to 10 |
| Composite powder* | 0.1 to 10 | 0.5 to 9 | 1 to 8 | 2 to 6 | 2 to 6 |
| Water | 0 to 40 | 0 to 25 | 0 to 10 | <1 | <0.5 |
| | Formula | Formula | Formula | Formula | Formula |
| | 46 | 47 | 48 | 49 | 50 |
| Hydrophobized metal | 5 to 20 | 5.5 to 18 | 6 to 15 | 6 to 10 | 6 to 10 |
| oxide powder | | | | | |
| Polyol | 40 to 94 | 45 to 92 | 60 to 90 | 65 to 88 | 65 to 88 |
| Disodium cetearyl | 0.1 to 15 | 1 to 12 | 2 to 11 | 5 to 10 | 5 to 10 |
| sulfosuccinate | | | | | |
| Composite powder* | 0.1 to 10 | 0.5 to 9 | 1 to 8 | 2 to 6 | 2 to 6 |
| Water | 0 to 40 | 0 to 25 | 0 to 10 | <1 | <0.5 |
| | Formula | Formula | Formula | Formula | Formula |
| | 51 | 52 | 53 | 54 | 55 |
| Hydrophobized metal | 5 to 20 | 5.5 to 18 | 6 to 15 | 6 to 10 | 6 to 10 |
| oxide powder | | | | | |
| Polyol | 40 to 94 | 45 to 92 | 60 to 90 | 65 to 88 | 65 to 88 |
| Sodium stearoyl | 0.1 to 15 | 1 to 12 | 2 to 11 | 5 to 10 | 5 to 10 |
| glutamate | | | | | |
| Composite powder* | 0.1 to 10 | 0.5 to 9 | 1 to 8 | 2 to 6 | 2 to 6 |
| Water | 0 to 40 | 0 to 25 | 0 to 10 | <1 | <0.5 |
| | Formula | Formula | Formula | Formula | Formula |
| | 56 | 57 | 58 | 59 | 60 |
| Silica Dimethicone | 5 to 20 | 5.5 to 18 | 6 to 15 | 6 to 10 | 6 to 10 |
| Silylate | | | | | |
| Polyol | 40 to 94 | 45 to 92 | 60 to 90 | 65 to 88 | 65 to 88 |
| Anionic emulsifier | 0.1 to 15 | 1 to 12 | 2 to 11 | 5 to 10 | 5 to 10 |
| Composite powder* | 0.1 to 10 | 0.5 to 9 | 1 to 8 | 2 to 6 | 2 to 6 |
| Water | 0 to 40 | 0 to 25 | 0 to 10 | <1 | <0.5 |
| | Formula | Formula | Formula | Formula | Formula |
| | 61 | 62 | 63 | 64 | 65 |
| Silica Dimethicone | 5 to 20 | 5.5 to 18 | 6 to 15 | 6 to 10 | 6 to 10 |
| Silylate | | | | | |
| Polyol | 40 to 94 | 45 to 92 | 60 to 90 | 65 to 88 | 65 to 88 |
| Disodium cetearyl | 0.1 to 15 | 1 to 12 | 2 to 11 | 5 to 10 | 5 to 10 |
| sulfosuccinate | | | | | |
| Composite powder* | 0.1 to 10 | 0.5 to 9 | 1 to 8 | 2 to 6 | 2 to 6 |
| Water | 0 to 40 | 0 to 25 | 0 to 10 | <1 | <0.5 |
| | Formula | Formula | Formula | Formula | Formula |
| | 66 | 67 | 68 | 69 | 70 |
| Silica Dimethicone | 5 to 20 | 5.5 to 18 | 6 to 15 | 6 to 10 | 6 to 10 |
| Silylate | | | | | |
| Polyol | 40 to 94 | 45 to 92 | 60 to 90 | 65 to 88 | 65 to 88 |
| Sodium stearoyl | 0.1 to 15 | 1 to 12 | 2 to 11 | 5 to 10 | 5 to 10 |
| glutamate | | | | | |
| Composite powder* | 0.1 to 10 | 0.5 to 9 | 1 to 8 | 2 to 6 | 2 to 6 |
| Water | 0 to 40 | 0 to 25 | 0 to 10 | <1 | <0.5 |
| | Formula | Formula | Formula | Formula | Formula |
| | 71 | 72 | 73 | 74 | 75 |
| Hydrophobized metal | 5 to 20 | 5.5 to 18 | 6 to 15 | 6 to 10 | 6 to 10 |
| oxide powder | | | | | |
| Glycerol | 40 to 94 | 45 to 92 | 60 to 90 | 65 to 88 | 65 to 88 |
| Anionic emulsifier | 0.1 to 15 | 1 to 12 | 2 to 11 | 5 to 10 | 5 to 10 |
| Composite powder* | 0.1 to 10 | 0.5 to 9 | 1 to 8 | 2 to 6 | 2 to 6 |
| Water | 0 to 40 | 0 to 25 | 0 to 10 | <1 | <0.5 |
| | Formula | Formula | Formula | Formula | Formula |
| | 76 | 77 | 78 | 79 | 80 |
| Hydrophobized metal | 5 to 20 | 5.5 to 18 | 6 to 15 | 6 to 10 | 6 to 10 |
| oxide powder | | | | | |
| Sorbitol | 40 to 94 | 45 to 92 | 60 to 90 | 65 to 88 | 65 to 88 |
| Anionic emulsifier | 0.1 to 15 | 1 to 12 | 2 to 11 | 5 to 10 | 5 to 10 |
| Composite powder* | 0.1 to 10 | 0.5 to 9 | 1 to 8 | 2 to 6 | 2 to 6 |
| Water | 10 to 40 | 10 to 30 | 10 to 30 | 15 to 30 | 15 to 30 |
| | Formula | Formula | Formula | Formula | Formula |
| | 81 | 82 | 83 | 84 | 85 |
| Hydrophobized metal | 5 to 20 | 5.5 to 18 | 6 to 15 | 6 to 10 | 6 to 10 |
| oxide powder | | | | | |
| Panthenol | 40 to 94 | 45 to 92 | 60 to 90 | 65 to 88 | 65 to 88 |
| Anionic emulsifier | 0.1 to 15 | 1 to 12 | 2 to 11 | 5 to 10 | 5 to 10 |
| Composite powder* | 0.1 to 10 | 0.5 to 9 | 1 to 8 | 2 to 6 | 2 to 6 |
| Water | 10 to 40 | 10 to 25 | 12 to 25 | 15 to 25 | 15 to 25 |
| | Formula | Formula | Formula | Formula | Formula |
| | 86 | 87 | 88 | 89 | 90 |
| Silica Dimethicone | 5 to 20 | 5.5 to 18 | 6 to 15 | 6 to 10 | 6 to 10 |
| Silylate | | | | | |
| Glycerol | 40 to 94 | 45 to 92 | 60 to 90 | 65 to 88 | 65 to 88 |
| Anionic emulsifier | 0.1 to 15 | 1 to 12 | 2 to 11 | 5 to 10 | 5 to 10 |
| Composite powder* | 0.1 to 10 | 0.5 to 9 | 1 to 8 | 2 to 6 | 2 to 6 |
| Water | 0 to 40 | 0 to 25 | 0 to 10 | <1 | <0.5 |
| | Formula | Formula | Formula | Formula | Formula |
| | 91 | 92 | 93 | 94 | 95 |
| Silica Dimethicone | 5 to 20 | 5.5 to 18 | 6 to 15 | 6 to 10 | 6 to 10 |
| Silylate | | | | | |
| Sorbitol | 40 to 94 | 45 to 92 | 60 to 90 | 65 to 88 | 65 to 88 |
| Anionic emulsifier | 0.1 to 15 | 1 to 12 | 2 to 11 | 5 to 10 | 5 to 10 |
| Water | 10 to 40 | 10 to 30 | 10 to 30 | 15 to 30 | 15 to 30 |
| | Formula | Formula | Formula | Formula | Formula |
| | 96 | 97 | 98 | 99 | 100 |
| Silica Dimethicone | 5 to 20 | 5.5 to 18 | 6 to 15 | 6 to 10 | 6 to 10 |
| Silylate | | | | | |
| Panthenol | 40 to 94 | 45 to 92 | 60 to 90 | 65 to 88 | 65 to 88 |
| Anionic emulsifier | 0.1 to 15 | 1 to 12 | 2 to 11 | 5 to 10 | 5 to 10 |
| Composite powder* | 0.1 to 10 | 0.5 to 9 | 1 to 8 | 2 to 6 | 2 to 6 |
| Water | 10 to 40 | 10 to 25 | 12 to 25 | 15 to 25 | 15 to 25 |
| | Formula | Formula | Formula | Formula | Formula |
| | 101 | 102 | 103 | 104 | 105 |
| Hydrophobized metal | 5 to 20 | 5.5 to 18 | 6 to 15 | 6 to 10 | 6 to 10 |
| oxide powder | | | | | |
| Glycerol | 40 to 94 | 45 to 92 | 60 to 90 | 65 to 88 | 65 to 88 |
| Disodium cetearyl | 0.1 to 15 | 1 to 12 | 2 to 11 | 5 to 10 | 5 to 10 |
| sulfosuccinate | | | | | |
| Composite powder* | 0.1 to 10 | 0.5 to 9 | 1 to 8 | 2 to 6 | 2 to 6 |
| Water | 0 to 40 | 0 to 25 | 0 to 10 | <1 | <0.5 |
| | Formula | Formula | Formula | Formula | Formula |
| | 106 | 107 | 108 | 109 | 110 |
| Hydrophobized metal | 5 to 20 | 5.5 to 18 | 6 to 15 | 6 to 10 | 6 to 10 |
| oxide powder | | | | | |
| Sorbitol | 40 to 94 | 45 to 92 | 60 to 90 | 65 to 88 | 65 to 88 |
| Disodium cetearyl | 0.1 to 15 | 1 to 12 | 2 to 11 | 5 to 10 | 5 to 10 |
| sulfosuccinate | | | | | |
| Composite powder* | 0.1 to 10 | 0.5 to 9 | 1 to 8 | 2 to 6 | 2 to 6 |
| Water | 10 to 40 | 10 to 30 | 10 to 30 | 15 to 30 | 15 to 30 |
| | Formula | Formula | Formula | Formula | Formula |
| | 111 | 112 | 113 | 114 | 115 |
| Hydrophobized metal | 5 to 20 | 5.5 to 18 | 6 to 15 | 6 to 10 | 6 to 10 |
| oxide powder | | | | | |
| Panthenol | 40 to 94 | 45 to 92 | 60 to 90 | 65 to 88 | 65 to 88 |
| Disodium cetearyl | 0.1 to 15 | 1 to 12 | 2 to 11 | 5 to 10 | 5 to 10 |
| sulfosuccinate | | | | | |
| Composite powder* | 0.1 to 10 | 0.5 to 9 | 1 to 8 | 2 to 6 | 2 to 6 |
| Water | 10 to 40 | 10 to 25 | 12 to 25 | 15 to 25 | 15 to 25 |
| | Formula | Formula | Formula | Formula | Formula |
| | 116 | 117 | 118 | 119 | 120 |
| Silica Dimethicone | 5 to 20 | 5.5 to 18 | 6 to 15 | 6 to 10 | 6 to 10 |
| Silylate | | | | | |
| Glycerol | 40 to 94 | 45 to 92 | 60 to 90 | 65 to 88 | 65 to 88 |
| Disodium cetearyl | 0.1 to 15 | 1 to 12 | 2 to 11 | 5 to 10 | 5 to 10 |
| sulfosuccinate | | | | | |
| Composite powder* | 0.1 to 10 | 0.5 to 9 | 1 to 8 | 2 to 6 | 2 to 6 |
| Water | 0 to 40 | 0 to 25 | 0 to 10 | <1 | <0.5 |
| | Formula | Formula | Formula | Formula | Formula |
| | 121 | 122 | 123 | 124 | 125 |
| Silica Dimethicone | 5 to 20 | 5.5 to 18 | 6 to 15 | 6 to 10 | 6 to 10 |
| Silylate | | | | | |
| Sorbitol | 40 to 94 | 45 to 92 | 60 to 90 | 65 to 88 | 65 to 88 |
| Disodium cetearyl | 0.1 to 15 | 1 to 12 | 2 to 11 | 5 to 10 | 5 to 10 |
| sulfosuccinate | | | | | |
| Composite powder* | 0.1 to 10 | 0.5 to 9 | 1 to 8 | 2 to 6 | 2 to 6 |
| Water | 10 to 40 | 10 to 30 | 10 to 30 | 15 to 30 | 15 to 30 |
| | Formula | Formula | Formula | Formula | Formula |
| | 126 | 127 | 128 | 129 | 130 |
| Silica Dimethicone | 5 to 20 | 5.5 to 18 | 6 to 15 | 6 to 10 | 6 to 10 |
| Silylate | | | | | |
| Panthenol | 40 to 94 | 45 to 92 | 60 to 90 | 65 to 88 | 65 to 88 |
| Disodium cetearyl | 0.1 to 15 | 1 to 12 | 2 to 11 | 5 to 10 | 5 to 10 |
| sulfosuccinate | | | | | |
| Composite powder* | 0.1 to 10 | 0.5 to 9 | 1 to 8 | 2 to 6 | 2 to 6 |
| Water | 10 to 40 | 10 to 25 | 12 to 25 | 15 to 25 | 15 to 25 |
| | Formula | Formula | Formula | Formula | Formula |
| | 131 | 132 | 133 | 134 | 135 |
| Hydrophobized metal | 5 to 20 | 5.5 to 18 | 6 to 15 | 6 to 10 | 6 to 10 |
| oxide powder | | | | | |
| Glycerol | 40 to 94 | 45 to 92 | 60 to 90 | 65 to 88 | 65 to 88 |
| Sodium stearoyl | 0.1 to 15 | 1 to 12 | 2 to 11 | 5 to 10 | 5 to 10 |
| glutamate | | | | | |
| Composite powder* | 0.1 to 10 | 0.5 to 9 | 1 to 8 | 2 to 6 | 2 to 6 |
| Water | 0 to 40 | 0 to 25 | 0 to 10 | <1 | <0.5 |
| | Formula | Formula | Formula | Formula | Formula |
| | 136 | 137 | 138 | 139 | 140 |
| Hydrophobized metal | 5 to 20 | 5.5 to 18 | 6 to 15 | 6 to 10 | 6 to 10 |
| oxide powder | | | | | |
| Sorbitol | 40 to 94 | 45 to 92 | 60 to 90 | 65 to 88 | 65 to 88 |
| Sodium stearoyl | 0.1 to 15 | 1 to 12 | 2 to 11 | 5 to 10 | 5 to 10 |
| glutamate | | | | | |
| Composite powder* | 0.1 to 10 | 0.5 to 9 | 1 to 8 | 2 to 6 | 2 to 6 |
| Water | 10 to 40 | 10 to 30 | 10 to 30 | 15 to 30 | 15 to 30 |
| | Formula | Formula | Formula | Formula | Formula |
| | 141 | 142 | 143 | 144 | 145 |
| Hydrophobized metal | 5 to 20 | 5.5 to 18 | 6 to 15 | 6 to 10 | 6 to 10 |
| oxide powder | | | | | |
| Panthenol | 40 to 94 | 45 to 92 | 60 to 90 | 65 to 88 | 65 to 88 |
| Sodium stearoyl | 0.1 to 15 | 1 to 12 | 2 to 11 | 5 to 10 | 5 to 10 |
| glutamate | | | | | |
| Composite powder* | 0.1 to 10 | 0.5 to 9 | 1 to 8 | 2 to 6 | 2 to 6 |
| Water | 10 to 40 | 10 to 25 | 12 to 25 | 15 to 25 | 15 to 25 |
| | Formula | Formula | Formula | Formula | Formula |
| | 146 | 147 | 148 | 149 | 150 |
| Silica Dimethicone | 5 to 20 | 5.5 to 18 | 6 to 15 | 6 to 10 | 6 to 10 |
| Silylate | | | | | |
| Glycerol | 40 to 94 | 45 to 92 | 60 to 90 | 65 to 88 | 65 to 88 |
| Sodium stearoyl | 0.1 to 15 | 1 to 12 | 2 to 11 | 5 to 10 | 5 to 10 |
| glutamate | | | | | |
| Composite powder* | 0.1 to 10 | 0.5 to 9 | 1 to 8 | 2 to 6 | 2 to 6 |
| Water | 0 to 40 | 0 to 25 | 0 to 10 | <1 | <0.5 |
| | Formula | Formula | Formula | Formula | Formula |
| | 151 | 152 | 153 | 154 | 155 |
| Silica Dimethicone | 5 to 20 | 5.5 to 18 | 6 to 15 | 6 to 10 | 6 to 10 |
| Silylate | | | | | |
| Sorbitol | 40 to 94 | 45 to 92 | 60 to 90 | 65 to 88 | 65 to 88 |
| Sodium stearoyl | 0.1 to 15 | 1 to 12 | 2 to 11 | 5 to 10 | 5 to 10 |
| glutamate | | | | | |
| Composite powder* | 0.1 to 10 | 0.5 to 9 | 1 to 8 | 2 to 6 | 2 to 6 |
| Water | 10 to 40 | 10 to 30 | 10 to 30 | 15 to 30 | 15 to 30 |
| | Formula | Formula | Formula | Formula | Formula |
| | 156 | 157 | 158 | 159 | 160 |
| Silica Dimethicone | 5 to 20 | 5.5 to 18 | 6 to 15 | 6 to 10 | 6 to 10 |
| Silylate | | | | | |
| Panthenol | 40 to 94 | 45 to 92 | 60 to 90 | 65 to 88 | 65 to 88 |
| Sodium stearoyl | 0.1 to 15 | 1 to 12 | 2 to 11 | 5 to 10 | 5 to 10 |
| glutamate | | | | | |
| Composite powder* | 0.1 to 10 | 0.5 to 9 | 1 to 8 | 2 to 6 | 2 to 6 |
| Water | 10 to 40 | 10 to 25 | 12 to 25 | 15 to 25 | 15 to 25 |
| | Formula | Formula | Formula | Formula | Formula |
| | 161 | 162 | 163 | 164 | 165 |
| Silica Dimethicone | 5 to 20 | 5.5 to 18 | 6 to 15 | 6 to 10 | 6 to 10 |
| Silylate | | | | | |
| Sorbitol and glycerol | 40 to 94 | 45 to 92 | 60 to 90 | 65 to 88 | 65 to 88 |
| Disodium cetearyl | 0.1 to 15 | 1 to 12 | 2 to 11 | 5 to 10 | 5 to 10 |
| sulfosuccinate | | | | | |
| Composite powder* | 0.1 to 10 | 0.5 to 9 | 1 to 8 | 2 to 6 | 2 to 6 |
| Water | 0.25 to 40 | 0.25 to 25 | 0.25 to 25 | 0.25 to 25 | 0.5 to 25 |
| | Formula | Formula | Formula | Formula | Formula |
| | 166 | 167 | 168 | 169 | 170 |
| Silica Dimethicone | 5 to 20 | 5.5 to 18 | 6 to 15 | 6 to 10 | 6 to 10 |
| Silylate | | | | | |
| Sorbitol and glycerol | 40 to 94 | 45 to 92 | 60 to 90 | 65 to 88 | 65 to 88 |
| Sodium stearoyl | 0.1 to 15 | 1 to 12 | 2 to 11 | 5 to 10 | 5 to 10 |
| glutamate | | | | | |
| Composite powder* | 0.1 to 10 | 0.5 to 9 | 1 to 8 | 2 to 6 | 2 to 6 |
| Water | 0.25 to 40 | 0.25 to 25 | 0.25 to 25 | 0.25 to 25 | 0.5 to 25 |

*Composite powder having the INCI name: Dimethicone/Vinyl Dimethicone Crosspolymer (and) Silica

The powdered, cosmetic compositions can contain other auxiliaries, care agents and additives. The proportion by weight of the further ingredients contained in the powdered compositions in addition to the components a) to e), in particular the further auxiliaries, care agents and additives contained in these compositions in the total weight of the powdered compositions is preferably less than about 10% by weight, preferably less as about 5% by weight, more preferably less than about 2% by weight and in particular less than about 1% by weight. The proportion by weight of these auxiliaries, care agents and additives in the total weight of the cosmetic compositions can be, for example, from about 0.01 to about 2% by weight, in particular from about 0.01 to about 0.5% by weight.

Preferred cosmetic compositions contain at least one oil body. The natural and synthetic cosmetic oil bodies include, for example, vegetable oils, liquid paraffin oils, isoparaffin oils and synthetic hydrocarbons, silicone oils and di-n-alkyl ethers having a total of from about 12 to about 36 carbon atoms, in particular from about 12 to about 24 carbon atoms.

A first preferred group of oil bodies are the vegetable oils. Examples of such oils are amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderflower seed oil, currant seed oil, jojoba oil, cocoa butter, linseed oil, macadamia nut oil, corn oil, almond oil, marula oil, evening primrose oil, olive oil, orange oil, palm oil, peach kernel oil, rapeseed oil, rice oil, sea buckthorn pulp oil, sea buckthorn seed oil, sesame oil, shea butter, soybean oil, sunflower oil, grapeseed oil, walnut oil, wheat germ oil, rosehip oil and the liquid portions of coconut oil. Also suitable, however, are other triglyceride oils such as the liquid portions of beef tallow as well as synthetic triglyceride oils.

Preferred oil bodies come from the group of silicone oils. The group of silicone oils includes in particular the dimethicones, to which the cyclomethicones are also to be counted, the amino-functional silicones and the dimethiconols. The dimethicones can be both linear and branched and cyclic or cyclic and branched. Suitable silicone oils or silicone gums are, in particular, dialkyl and alkylaryl siloxanes, such as dimethylpolysiloxane and methylphenylpolysiloxane, and also their alkoxylated, quaternized or else anionic derivatives. Preference is given to cyclic and linear polydialkylsiloxanes, their alkoxylated and/or aminated derivatives, dihydroxypolydimethylsiloxanes and polyphenylalkylsiloxanes.

Ester oils, that is, esters of C₆-C₃₀ fatty acids with C₂-C₃₀ fatty alcohols, preferably monoesters of fatty acids with alcohols having from about 2 to about 24 carbon atoms such as isopropyl myristate (Rilanit® IPM), isononanoic acid C16-18 alkyl ester (Cetiol® SN), 2-ethylhexyl palmitate (Cegesoft® 24), stearic acid-2-ethylhexyl ester (Cetiol® 868), cetyl oleate, glycerol tricaprylate, cocofatty alcohol caprate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are more preferred oil bodies.

With regard to the cosmetic effect, cosmetic compositions have proved to be advantageous in which the proportion by weight of the oil body in the total weight of the composition is from about 0.01 to about 5% by weight, preferably from about 0.02 to about 4% by weight and in particular from about 0.05 to about 2% by weight.

The agent can contain a protein hydrolyzate and/or one of its derivatives as a further care substance. Protein hydrolyzates are product mixtures which can be obtained by acid, alkaline or enzymatically catalyzed degradation of proteins. The term protein hydrolyzates is also understood to mean total hydrolyzates and also individual amino acids and their derivatives and mixtures of different amino acids. The molecular weight of the protein hydrolysates lies between about 75, the molecular weight of glycine, and about 200,000, preferably the molecular weight is from about 75 to about 50,000 and most preferably from about 75 to about 20,000 daltons.

The powdered composition can further contain at least one vitamin, a provitamin, a vitamin precursor and/or one of their derivatives as a care substance. In this case, those vitamins, provitamins and vitamin precursors are preferred, which are usually assigned to the groups A, B, C, E, F and H.

The powdered compositions can further contain at least one plant extract, but also mono- or oligosaccharides and/or lipids as a care substance.

The powdered compositions can contain at least one UV filter as a care substance. The UV filters are present in the powdered compositions usually in amounts of from about 0.01 to about 5% by weight, based on the powdered composition. Amounts of from about 0.1 to about 2.5% by weight are preferred.

The powdered compositions can be packaged in any containers, as long as it is ensured that the mechanical loading of the powder during the removal of the composition does not lead to liquefaction. For example, cups, bottles or tetra packs are suitable, wherein the container can be configured, for example, with a bulk and dosing device.

A third subject of the present application is therefore the use of a powdered cosmetic composition for temporary deformation of keratin-containing fibers, in particular human hair. When using the powdered composition for the temporary deformation of keratinic fibers, the desired amount of the powdered composition is preferably first removed from the container. The composition can in this case be applied directly to the keratinic fiber to be treated or, however, for example, to the hand. In the first case, the powder applied directly on the keratin fiber can be subjected to a mechanical load, for example, by employing the hands, whereby the liquid, aqueous phase is released directly on the fiber. If the powdered composition is first applied to the hand, then it can first be carefully distributed in the hair and only then again be subjected to greater mechanical stress, for example, by targeted massaging of the powder into the hair. This releases the liquid, aqueous phase on the hair.

It is of course also possible to rub the powdered composition on hand and only apply the resulting liquid or pasty agent on the keratinic fiber. Of course, the powdered composition can also be applied with an aid, such as a brush, a sponge, a cloth, a brush or a comb.

With regard to further preferred embodiments of the use, what has been said about the powdered cosmetic compositions applies mutatis mutandis.

A further subject of the present application is a method for the temporary deformation of keratin-containing fibers, in particular human hair, in which the keratinic fibers are acted upon with a cosmetic composition as contemplated herein and temporarily fixed in its shape, exemplified in that a plastically deformable mass is formed from the cosmetic composition by the action of a force before, during or after application to the keratinic fibers.

Finally, a method is also claimed for the production of the powdered cosmetic composition, containing, based on its total weight,
a) from about 5 to about 20% by weight of hydrophobized metal oxide powder
b) from about 40 to about 94% by weight of organic polyol
c) from about 0.1 to about 15% by weight of emulsifier
d) from about 0.1 to about 10% by weight of a composite powder, including
(A) powder particles of a silicone elastomer and
(B) particles of silicon dioxide,
wherein the particles of silicon dioxide (B) are immobilized on the surface of the powder particles of the silicone elastomer (A), and
e) from 0 to about 40% by weight of water comprising the steps
i) mixing the constituents b) and optionally d);
ii) introducing the hydrophobized metal oxide powder a) into the mixture resulting from step i) and remixing, preferably for a period of time of from about 1 to about 120 seconds, preferably from about 10 to about 60 seconds, to form a powder;
iii) introducing the preferably powdered emulsifier c), the composite powder d) and optionally the water e) into the powder produced in step ii) and remixing, preferably for a period of from about 1 to about 120 seconds, preferably from about 10 to about 60 seconds.

Preferably, steps ii) and iii) are performed with the aid of a tumble mixer.

With regard to further preferred embodiments of the method, what has been said about the powdered cosmetic compositions applies mutatis mutandis.

### EXAMPLES

The powdered styling agents E1 to V7 were prepared as described below (specifications in % by weight):

| Raw materials | E1 |
|---|---|
| Aerosil ® R 202 ¹⁾ | 7.5 |
| Sorbitol²⁾ | 33 |
| Glycerol | 35 |
| Eumulgin SG ³⁾ | 9 |
| Dow Corning 9701 Cosmetic | 3 |
| Powder | |

¹⁾ Fumed silica aftertreated with polydimethylsiloxane (INCI name: Silica Dimethicone Silylate)
²⁾Sorbitol, 70% in water
³⁾ Sodium Stearoyl Glutamate (INCI)
4) Dimethicone/Vinyl Dimethicone Crosspolymer (and) Silica (INCI)

All constituents except Aerosil® R 202, Dow Corning 9701 Cosmetic Powder and the emulsifier were mixed in a vessel. This liquid was mixed with the hydrophobized silicon dioxide powder Aerosil® R 202 S. In each case, a stable powder had formed after a stirring time of 30 to 45 seconds. Subsequently, the obtained powder was placed in a tumble mixer and mixed with the emulsifier and the composite powder. The finished styling powder thus obtained was filled into polyethylene bottles.

Powdered styling agents which contained instead of 3% by weight composite powder with the INCI name Dimethicone/Vinyl Dimethicone Crosspolymer (and) Silica 3% by weight maltitol or xylitol powder, powdered cocoa butter or powdered polymers such as Advantage S (INCI: Vinyl Caprolaetain/VP/Dimethylaminoethyl Methacrylate Copolymer) or Sepinov EMT10 (INCI: Hydroxyethyl Acrylate/Sodium Acryloyidimethyl Taurate Copolymer), showed no appreciable improvement in haptics on the skin, in particular no reduced stickiness on the hand, and/or were unstable.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A powdered cosmetic composition comprising, based on its total weight
a) from about 5 to about 20% by weight of hydrophobized metal oxide powder,
b) from about 40 to about 94% by weight of organic polyol,
c) from about 0.1 to about 15% by weight of emulsifier,
d) from about 0.1 to about 10% by weight of composite powder, comprising
(A) powder particles of a silicone elastomer and
(B) particles of silicon dioxide,
wherein the particles of silicon dioxide (B) are immobilized on the surface of the powder particles of the silicone elastomer (A), and
e) from 0 to about 40% by weight of water.

2. The composition according to claim 1, comprising a hydrophobized silicate as the hydrophobized metal oxide powder.

3. The composition according to claim 1, comprising a fumed silica aftertreated with polydimethylsiloxane as the hydrophobized metal oxide powder.

4. The composition according to claim 1, comprising the organic polyol, based on its total weight, in amounts of from about 45 to about 92% by weight.

5. The composition according to claim 1, comprising at least one compound from the group glycerol, sorbitol and panthenol as the organic polyol.

6. The composition according to claim 1, comprising a mixture of glycerol and sorbitol as the organic polyol.

7. The composition according to claim 1, comprising as an emulsifier an emulsifier solid at room temperature of 20° C.

8. The composition according to claim 1, comprising, as a composite powder, a powder having the INCI name Dimethicone/Vinyl Dimethicone Crosspolymer (and) Silica.

10. A method for the temporary deformation of keratin-containing fibers, in which the keratinic fibers are acted upon by a cosmetic composition according to claim 1 and temporarily fixed in their form, wherein a plastically deformable mass is formed from the cosmetic composition by action of a force before, during or after application to the keratinic fibers.

11. The composition according to claim 1, comprising the organic polyol, based on its total weight, in amounts of from about 65 to about 88% by weight.

12. The composition according to claim 1, comprising as an emulsifier an anionic emulsifier solid at room temperature of 20° C.

13. The composition according to claim 1, comprising as an emulsifier at least one emulsifier selected from the group of cetearyl sulfosuccinates or stearoyl glutamate.
